(19) United States
(12) Patent Application Publication
(10) Pub. No.: US 2021/0374709 A1
(43) Pub. Date: Dec. 2, 2021

(54) CREATION OF RESTRICTED MOBILE ACCOUNTS

(71) Applicant: Apple Inc., Cupertino, CA (US)

(72) Inventors: Akila Suresh, San Jose, CA (US); Muhammad Noman, Santa Clara, CA (US); Richard W. Heard, San Francisco, CA (US); Katie M. McIndoe, San Jose, CA (US)

(73) Assignee: Apple Inc., Cupertino, CA (US)

(21) Appl. No.: 17/031,685

(22) Filed: Sep. 24, 2020

Related U.S. Application Data
(60) Provisional application No. 63/032,500, filed on May 29, 2020.

(51) Int. Cl. G06Q 20/22; G06Q 20/10; G06Q 20/32; G06Q 20/42; G06Q 20/38; G06Q 40/02; G06Q 20/40; G06Q 30/00; G06Q 20/36; G06F 16/22; H04L 29/06

(52) U.S. Cl. CPC G06Q 20/2295; G06Q 20/108; G06Q 20/3223; G06Q 20/3263; G06Q 20/42; G06Q 20/3829; G16H 10/60; G06Q 20/4014; G06Q 30/0185; G06Q 20/3674; G06F 16/2282; H04L 63/0853; G06Q 2220/00; G06Q 40/02

(57) ABSTRACT

A first user device may request a provisioning system to create a restricted mobile payment account on a second user device. The provisioning system may send a registration notification to the second user device to trigger registration of the restricted mobile payment account. The provisioning system may also cause creation of the restricted mobile payment account based on account information received with the request from the first user device.

### CROSS-REFERENCE TO RELATED APPLICATION

The present application claims the benefit of and priority to U.S. Provisional Application No. 63/032,500, filed May 29, 2020, entitled “CREATION OF RESTRICTED MOBILE ACCOUNTS.” The entire contents of which are incorporated herein by reference for all purposes. This application is related to U.S. Provisional Application No. 63/032,399, filed May 29, 2020, entitled “CONFIGURING AN ACCOUNT FOR A SECOND USER IDENTITY.” The full disclosure of which is incorporated by reference herein in its entirety for all purposes.

### BACKGROUND

Portable electronic devices such as smartphones and smartwatches may use digital wallets to host applications and store relevant credential information. A portable electronic device may use its digital wallet to selectively share parts of the credential information during contactless transactions (e.g., contactless payment at a payment terminal, contactless debiting of a transit fair, etc.) and during peer-to-peer money transfers. In some cases, the digital wallet may be linked to one or more payment accounts hosted by various third parties.

### BRIEF SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a computer-implemented method, including receiving, from a first user device associated with a first user account, a request to establish a restricted mobile payment account on behalf of a second user account. The restricted mobile payment account to be a subaccount of a primary mobile payment account of the first user account. The request may include user account information that is associated with the second user account. The computer-implemented method also includes determining that the second user account is included in a user account group with the first user account based at least in part on the user account information. The computer-implemented method also includes sending a registration notification to a second user device to trigger registration of the restricted mobile payment account with a payment service, the second user device associated with the second user account. The computer-implemented method also includes receiving, from the second user device and based at least in part on the registration notification, a registration request that includes a unique user account identifier of the second user account. The computer-implemented method also includes causing creation of the restricted mobile payment account based at least in part on the user account information and the unique user account identifier of the second user account. The computer-implemented method also includes registering the first user account to receive transaction activity notifications corresponding to transactions of the restricted mobile payment account. Other embodiments of this aspect include corresponding computer systems, apparatus, and computer programs recorded on one or more computer storage devices, each configured to perform the actions of the methods.

Another general aspect includes a computer-implemented method including detecting that an age associated with a first user account meets or exceeds an age threshold. The first user account associated with a restricted mobile payment account that is a subaccount of a primary account of a second user account. The computer-implemented method may also include presenting a conversion option at a user interface of the user device based at least in part on detecting that the age associated with the first user account meets or exceeds the age threshold, the conversion option enabling conversion of the restricted mobile payment account to a new primary mobile payment account of the first user account. The computer-implemented method also includes, responsive to a selection of the conversion option at the user interface, communicating with a payment service to verify an identity of a user of the first user account.

The computer-implemented method also includes receiving a communication indicating successful creation of the new primary mobile payment account. Other embodiments of this aspect include corresponding computer systems, apparatus such as a user device, and computer programs recorded on one or more computer storage devices, each configured to perform the actions of the methods.

### BRIEF DESCRIPTION OF THE DRAWINGS

FIG. 1 illustrates a block diagram and a flowchart showing an example process for establishing restricted mobile payment accounts for use on user devices, according to at least one example.

FIG. 2 illustrates a block diagram showing an example architecture or system for establishing restricted mobile payment accounts for use on user devices, according to at least one example.

FIG. 3 illustrates user interfaces for establishing restricted mobile payment accounts for use on user devices, according to at least one example.

FIG. 4 illustrates user interfaces for establishing restricted mobile payment accounts for use on user devices, according to at least one example.

FIG. 5 illustrates user interfaces for establishing restricted mobile payment accounts for use on user devices, according to at least one example.

FIG. 6 illustrates user interfaces for establishing restricted mobile payment accounts for use on user devices, according to at least one example.

FIG. 7 illustrates user interfaces for establishing restricted mobile payment accounts for use on user devices, according to at least one example.

FIG. 8 illustrates user interfaces for establishing restricted mobile payment accounts for use on user devices, according to at least one example.

FIGS. 9A-9C illustrate a sequence diagram showing an example process for establishing restricted mobile payment accounts for use on user devices, according to various examples.

FIGS. 10 illustrates a sequence diagram showing an example processes for sharing notifications relating to transactions of restricted mobile payment accounts, according to at least one example.

FIGS. 11A and 11B illustrate a sequence diagram showing an example process for converting a restricted mobile payment account to a primary payment account, according to at least one example.

FIG. 12 illustrates a table 1200 showing an example process for sharing data with and among mobile payment accounts, according to at least one example.

FIG. 13 illustrates a flowchart showing an example process for establishing restricted mobile payment accounts for use on user devices, according to at least one example.

FIG. 14 illustrates a flowchart showing an example process for converting a restricted mobile payment account to a primary payment account, according to at least one example.

FIG. 15 illustrates a simplified block diagram depicting an example architecture for implementing the techniques described herein, according to at least one example.

### DETAILED DESCRIPTION

In the following description, various examples will be described. For purposes of explanation, specific configurations and details are set forth in order to provide a thorough understanding of the examples. However, it will also be apparent to one skilled in the art that the examples may be practiced without the specific details. Furthermore, well-known features may be omitted or simplified in order not to obscure the example being described.

Examples of the present disclosure are directed to, among other things, methods, systems, devices, and computer-readable storage media for establishing restricted mobile payment accounts. The restricted mobile payment accounts may enable users to use their mobile electronic devices (e.g., smartphones, tablets, smartwatches, etc.) to conduct contactless transactions (e.g., wireless communication with near field communication devices), which may include payment for goods and services at point-of-sale terminals, peer-to-peer transactions such as transferring money within a messaging application, and the like. A mobile payment account may be restricted in the sense that it is a subaccount of a primary payment account and is subject to control of an owner of the primary payment account (e.g., restrictions of transaction types, transfer amounts, parties who can send and receive funds, etc.). Because the restricted mobile payment account is a subaccount of the primary payment account, the restricted mobile payment account may be established without the rigorous identity validation steps typically performed when an issuing entity such as a bank establishes a new account. For example, the owner may use her mobile electronic device to create a restricted mobile payment account and configure her child's electronic device to use the account without the owner or the child having to perform identity validation or, in some cases, even interact with the child's electronic device. This may enable creation, funding, and activation of the restricted mobile payment account in a frictionless and, in some cases, almost instantaneous manner.

In some examples, when a child becomes an adult (e.g., turns 18 years of age in the United States), she may be able to open her own primary payment account, which may include converting a restricted mobile payment account to the primary payment account. To do so, the child (now an adult) would be subject to identity validation and other fraud/security measures exacted by a service provider the offers the payment service and/or an issuing entity at which the account resides. Conversion to the primary payment account effectively breaks the ties with the old primary payment account. After conversion, the old primary account owner no longer can place restrictions on the child's account. Conversion also enables the child to retain an account transaction history that includes activity on the restricted mobile payment account.

Turning now to a particular example, a provisioning system is described that enables an adult parent on a first mobile phone to request creation of a restricted mobile payment account on a second mobile phone for her child. The restricted mobile payment account is created as a subaccount of, and that is associated with, a primary payment account of the parent. To begin, the parent uses the first mobile phone to identify her child (e.g., select from a list of users or input her child's username) and requests the provisioning system to create the restricted mobile payment account. The provisioning system may check to see if the child already has an account and whether the parent's payment account is active. The provisioning system may also check to see if an existing trusted relationship exists between the parent and the child. For example, the provisioning system or another system offered by a service provider that operates the provisioning system may provide a service that maintains trusted relationships, and this service may be relied upon to determine whether the parent and the child are part of a trusted group of accounts (e.g., a trusted family circle of accounts). If so, the provisioning system may continue to set up the restricted mobile payment account by causing the second mobile phone to register with the provisioning system and any other payment systems, communicate with an issuing entity to open an account, and perform any fraud checks. The first mobile phone (or at least the parent's user account) may also be registered to receive notifications about transaction activity of the restricted mobile payment account.

The systems, devices, and techniques described herein provide several technical advantages that improve the security of establishing payment accounts, and protect user privacy. For example, reliance on a previously vetted trusted circle of user accounts ensures that the user accounts meet a minimum standard of user account verification, at least for purposes of creating a restricted mobile payment account. Thus, non-adult members of the trusted circle may not need to share their user account information for additional verification purposes, thereby limiting the spread of personal information and maintaining privacy and security of non-adult members.

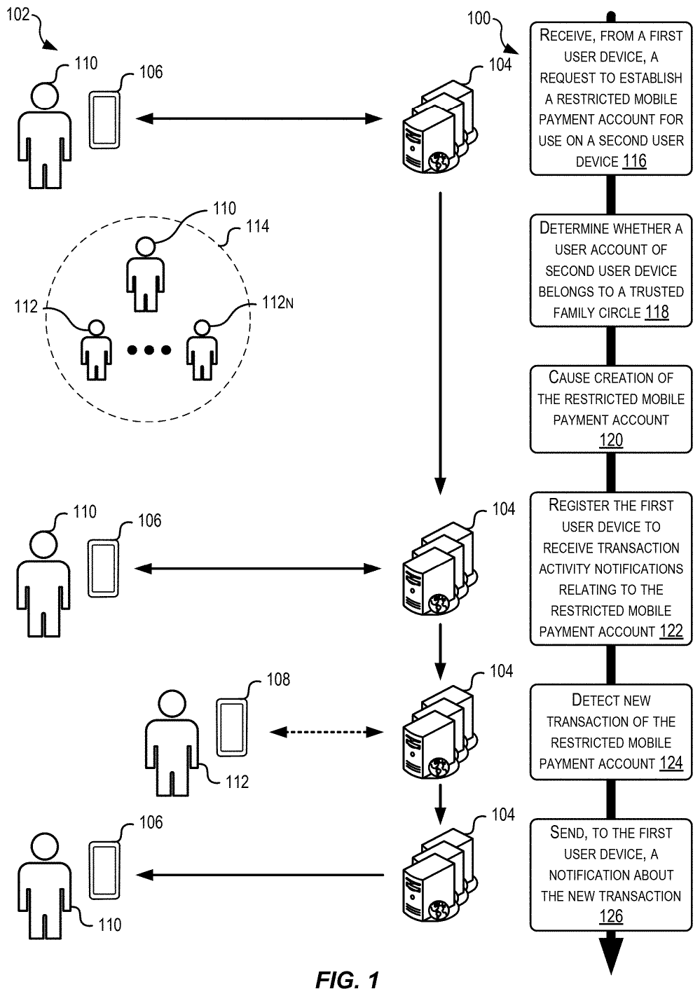

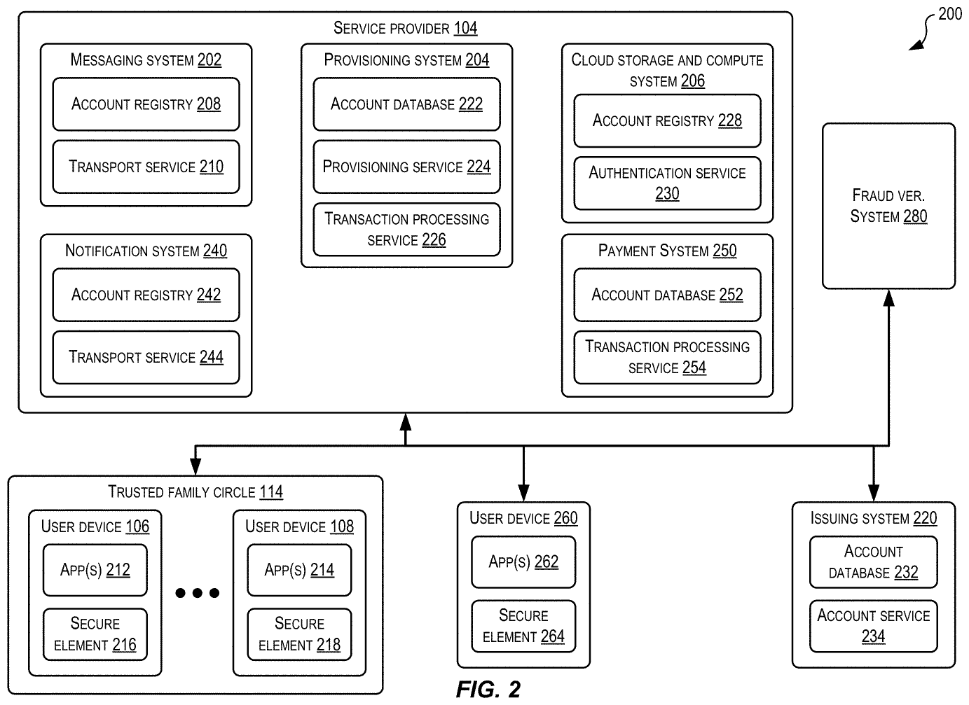

Turning now to the figures, FIG. 1 illustrates a block diagram 102 and a flowchart showing a process 100 for establishing restricted mobile payment accounts for use on user devices, according to at least one example. The diagram 102 includes a service provider 104. As described in further detail with respect to FIG. 2, the service provider 104 is any suitable combination of computing devices such as one or more server computers, which may include virtual resources, capable of performing the functions described with respect to the service provider. Generally, the service provider 104 is configured to manage aspects of establishing mobile payment accounts, including restricted and non-restricted (e.g., primary) accounts, processing certain types of payment requests, and maintaining transaction history for certain transactions.

The diagram 102 also includes an unrestricted user device 106 operated by an owner user 110 and a restricted user device 108 operated by a restricted user 112. The unrestricted user device 106 and the restricted user device 108 may be the same type of user device, but different numbers are used here to designate that their respective functions differ depending on whether the device is used to manage a primary account and request creation of a restricted mobile payment account, e.g., the unrestricted user device 106, or is the device for which account creation is requested, e.g., the restricted user device 108. The user devices 106 and 108 are any suitable electronic user device capable of communicating with other electronic devices over a network such as the Internet, a cellular network, or any other suitable network. In some examples, the user devices 106 and 108 may be a smartphone, mobile phone, smart watch, tablet, or other portable or non-portable electronic user device on which specialized applications can operate. The user devices 106 and 108 may include digital wallets, which as described herein, may be stored in secure elements and may host one or more payment accounts each with its own payment instrument. The unrestricted user device 106 may be uniquely associated with the owner user 110 (e.g., via an account used to log in to the unrestricted user device 106), and the restricted user device 108 may be uniquely associated with the restricted user 112 in the same manner. In some examples, the restricted user device 108 may also be associated with the unrestricted user device 106 via a link with the account of the owner user 110 (e.g., part of a family or trusted family circle 114 of trusted user accounts). This link may make the restricted user device 108 a trusted user device with respect to the unrestricted user device 106. For example, the trusted family circle 114 of accounts depicted include the owner user 110 (depicted largest) and one or more restricted users 112-112n. In some examples, the owner user 110 may use her user account to manage aspects of the accounts of the restricted users 112-112n.

FIGS. 1, 9A-9C, 10, 11A, 11B, 13, and 14 illustrate example flow diagrams showing processes 100, 900, 1000, 1100, 1300, and 1400 according to at least a few examples. These processes, and any other processes described herein, are illustrated as logical flow diagrams, each operation of which represents a sequence of operations that can be implemented in hardware, computer instructions, or a combination thereof In the context of computer instructions, the operations may represent computer-executable instructions stored on one or more non-transitory computer-readable storage media that, when executed by one or more processors, perform the recited operations. Generally, computer-executable instructions include routines, programs, objects, components, data structures and the like that perform particular functions or implement particular data types. The order in which the operations are described is not intended to be construed as a limitation, and any number of the described operations can be combined in any order and/or in parallel to implement the processes.

Additionally, some, any, or all of the processes described herein may be performed under the control of one or more computer systems configured with specific executable instructions and may be implemented as code (e.g., executable instructions, one or more computer programs, or one or more applications) executing collectively on one or more processors, by hardware, or combinations thereof. As noted above, the code may be stored on a non-transitory computer-readable storage medium, for example, in the form of a computer program including a plurality of instructions executable by one or more processors.

The process 100 may begin at 116 by the service provider 104 receiving, from a first user device (e.g., the unrestricted user device 106), a request to establish a restricted mobile payment account for use on a second user device (e.g., the restricted user device 108). The request may be initiated in a digital wallet application on the unrestricted user device 106, within a settings application on the unrestricted user device 106 (e.g., an option for managing aspects of the trusted family circle 114), or in any other suitable manner (e.g., via a web application, third-party application, etc.). The request may include user account information of the restricted user 112.

At 118, the process 100 includes the service provider 104 determining whether a user account of the second user device (e.g., the restricted user device 108) belongs to a trusted circle (e.g., the trusted family circle 114). If the user account does belong to the trusted family circle 114, the service provider 104 may rely on previously-established and verified identities as part of establishing the new account. If not, the restricted user 112 may have to perform independent identity verification.

At 120, the process 100 includes the service provider 104 causing creation of the restricted mobile payment account. This may be used performed using the user account information of the restricted user 112 and, in some examples, at least some information input by the owner user 110. Creating the account may include communicating with an issuing entity to create the account, which is associated with a primary payment account of the owner user 110.

At 122, the process 100 includes the service provider 104 registering the first user device (e.g., the unrestricted user device 106) to receive transaction activity notifications relating to the restricted mobile payment account. At this point, the unrestricted user device 106 may also be used by the owner user 110 to establish account restrictions on the restricted mobile payment account.

At 124, the process 100 includes the service provider 104 detecting a new transaction of the restricted mobile payment account. For example, depending on the restrictions/permissions established by the owner user 110, this may include a contactless payment transaction, a peer-to-peer payment transaction, an in-app payment, etc.

At 126, the process 100 includes the service provider 104 sending, to the first user device (e.g., the unrestricted user device 106), a notification about the new transaction. This may be in the form of a push notification, email message, instant message alert, or shared in any other suitable manner.

FIG. 2 illustrates a block diagram showing an example architecture or system 200 for establishing restricted mobile payment accounts for use on user devices, according to at least one example. The system 200 may generally be used to manage payment processing, establish new accounts, and the like. In particular, the system 200 may be used to establish restricted mobile payment accounts. As such, the system 200 includes a few elements introduced in FIG. 1. In particular, the system 200 includes the service provider 104, the unrestricted user device 106, and the restricted user device 108. The arrows between elements of the system 200 generally indicate that these element are communicatively coupled, either via a wired network connection, a wireless connection, or in any other suitable manner. However, the arrows should not be viewed as limited because at least some elements may communicate with each other, despite there not being arrows between them.

Beginning with the service provider 104, the service provider 104 may be operated by the same entity that sells the user device 106 and 108. In this manner, the user devices 106 and 108 may generally operate in an ecosystem hosted by the service provider 104. The service provider 104 includes a messaging system 202, a provisioning system 204, a cloud storage and compute system 206, a notification system 240, and a payment system 250. The system 200 may also include an issuing system 220, which is an entity that issues the payment accounts described herein, and a fraud verification system 280, which is an entity that performs certain types of fraud checks when new accounts are created.

The user devices 106 and 108 are included in the trusted family circle 114. The system 200, however, also includes restricted user device 260. Each device, 106, 108, and 260 respectively includes one or more app(s) 212, 214, 262 and a secure element 216, 218, and 264. The secure elements 216, 218, and 264 are used to store credentials, metadata, images, and other such information relating to the payment instruments and payment accounts. In some examples, the secure elements 216, 218, and 264 provide a platform for conducting contactless transactions with payment terminals, entry terminals, and the like.

The apps 212, 214, and 264 may be any suitable computer program or software application developed to run on a mobile device. The apps 212, 214, and 264 may be pre-loaded on the user devices 106, 108, and 260 (e.g., a messaging application, a mobile wallet application, an email application, etc.) and/or may be downloaded from an application store. In some examples, at least some of the apps available in the application store may be developed by third-parties, e.g., a party other than the developer of the pre-loaded applications and/or the operating system of the user devices 106, 108, and 260. In some examples, these “third-party apps” may enable access to certain credentials. For example, a third-party banking application on the user device 106 may be used to load a credit card credential into the secure element 216, and share the credential with the user device 108.

Turning now to the details of the service provider 104, the messaging system 202 is generally configured to host messaging applications (e.g., one of the apps 212) on the user devices 106, 108, and 260. The messaging system 202 includes an account registry 208 and a transport service 210. The account registry 208 may be used to store registered device identifiers (e.g., phone numbers, email addresses, etc.) at which users may receive messages and from which users may send messages. In some examples, the device identifiers may be associated with the user accounts used to log in to the user devices. The transport service 210 is generally configured to enable transportation of messages by and between user devices that include the messaging applications. For example, the transport service 210 may enable messages to be sent between the user device 106 and the user device 108. In some examples, the transport service 210 may include functionality to enable end-to-end encryption of messages sent between the user devices 106, 108, 260, and other devices. For example, a messaging application on the user device 106 may be used to send an encrypted message including a payment request to the user device 108. In some examples, the messaging applications on the user devices encrypt and decrypt messages using keys that are not known to the messaging system 202 and/or the service provider 104.

Turning now to the provisioning system 204, the provisioning system 204 includes an account database 222, a provisioning service 224, and a transaction processing service 226. Generally, the account database 222 may be used for storing account information for users and their respective devices that interact with the provisioning system 204. The provisioning service 224 may be configured to perform operations described here with respect to the flowcharts relating to establishing payment accounts on user devices. Thus, the provisioning service 224 may include functionality to communicate with the user devices 106, 108 and 260 other elements in the service provider 104, the fraud verification system 280, and the issuing system 220. The transaction processing service 226 may be used to process peer-to-peer type payment requests, such as those requested using messaging applications and transported via the messaging system 202.

The payment system 250 may be configured to process certain types of payment such as contactless point of sale transactions. Thus, like the provisioning system 204, the payment system 250 may include an account database 252, which may include similar information as the account database 222, and a transaction processing service 254, which may be configured similarly as the transaction processing service 226.

The notification system 240 may include an account registry 242 and a transport service 244. Generally, the notification system 240 may be used to send push notifications to the user devices 106, 108, and 260. For example, when a new restricted mobile payment account is created for a user, a record may be stored in the account registry 242. The record may identify the other user accounts to receive notifications about account activity on the restricted mobile payment account. For example, this may include an owner and/or the parents. The transport service 244 may be used to push the notifications to the relevant user devices.

Generally, the cloud storage and compute system 206 enables users to store data such as documents, photos, videos, etc. on remote servers, provides means for wirelessly backing up data on user devices, and provides data syncing between user devices. The cloud storage and compute system 206 includes an account registry 228 and an authentication service 230. The account registry 228 is used to store account information including registered device identifiers (e.g., phone numbers, email addresses, etc.) for users that utilize the cloud storage and compute system 206. In some examples, an account with the cloud storage and compute system 206 may be needed to initialize the user devices 106, 108, and 260. In some examples, at least a portion of the account information in the account registry 228 is the same as at least a portion of the account information in the account registry 208. For example, a user may use the same email address (e.g., username) for both systems and may associate the same device identifiers for both systems. In this manner, the same user devices may be used to receive messages, per the account registry 208, and access remote files, per the account registry 228. In some examples, the account registry 228 may be used as a point of truth for authenticating user/user devices requesting creation of new accounts. The authentication service 230 may perform the function of authentication, as described herein.

Generally, the issuing system 220 may be operated by an entity other than the entity that operates the service provider 104. In particular, the issuing system 220 may be an example of an external computer system that hosts payment accounts. For example, the issuing system 220 may be associated with a bank that issues a credit card or a virtual cash card. The issuing system 220 includes an account database 232 and an account service 234. Generally, the account database 232 is used to store account information that is specific to the issuing system 220. For example, this may be a user's login information for a bank account that is owned by the entity that operates the issuing system 220, account numbers, account balances, record of transactions, etc. The account information may also identify associations between user devices having third-party applications published by the issuing system 220 and users whose account information is stored in the account database 232.

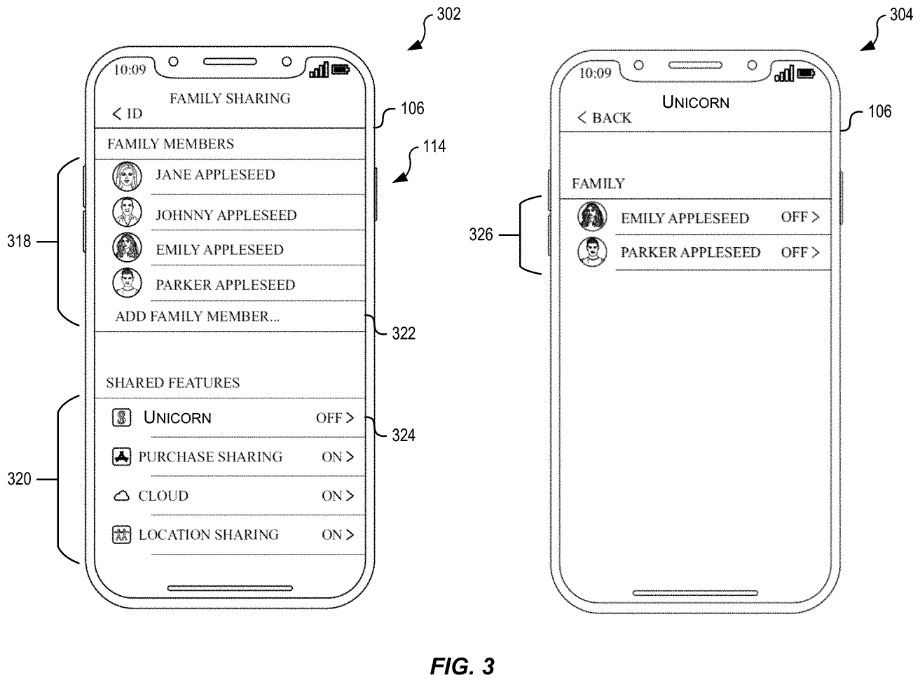

FIGS. 3-6 illustrate user interfaces for establishing restricted mobile payment accounts for use on user devices, according to various examples. The user interfaces in FIGS. 3-6 in particular may be presented on the unrestricted user device 106 and may represent the views presented to a user as part of new restricted account creation and management.

FIGS. 3-6 illustrate user interfaces 302-216 presented on the unrestricted user device 106. In particular, the user interface 302 is presented within a settings application on the unrestricted user device 106, the device of the owner user 110. From the settings application, the user may be able to manage permissions relating to the trusted family circle 114. For example, the user interface 302 includes a list of family members 318 and a list of shared features 320. In the user interface 302, the list of family members 318 includes four members. A trusted family circle may include at least three roles: an owner (e.g., an owner user), another adult (e.g., a managing user or managing participant), and a participant (e.g., a restricted user). In this example, Jane may be the owner, Johnny may be the other adult, and children, Emily and Parker may be the participants. The owner has the highest number of permissions, followed by the other adult, and the participants. To add a user to the trusted family circle 114, the add family member button 322 may be selected. Adding a family member may include inputting login credentials for the user (or creating a new user account) and associating the account with the trusted family circle 114.

Members of the trusted family circle 114 identified in the list of family members 318 may be entitled to share certain features such as purchase sharing, cloud storage resource sharing, location sharing, media and entertainment sharing, etc. depicted in the list of shared features 320. The techniques described herein relate to the process of creating mobile payment accounts for restricted users (e.g., Emily and/or Parker). To do so, the owner user (e.g., Jane) may select the “Unicorn” button 324, which is currently shown as turned “off”. Selection of this button 324, may cause the unrestricted user device 106 to present the user interface 304. The user interface 304 presents a list of eligible users 326 for which restricted mobile payment accounts can be created. The list of eligible users 326 in particular includes Emily and Parker, the two children (e.g., not yet 18 years of age) in the trusted family circle 114.

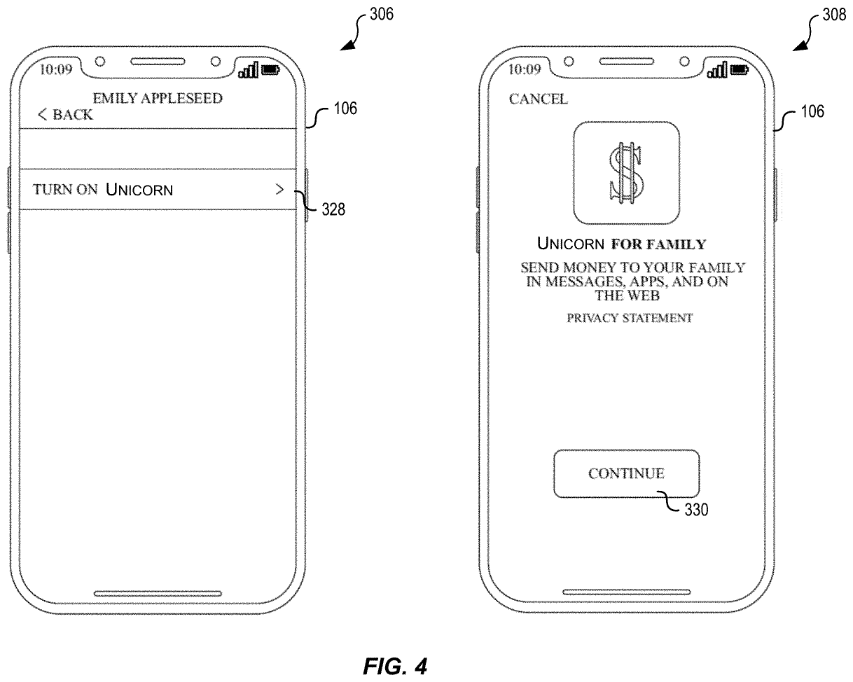

Selection of one of the names, Emily or Parker, in the user interface 304 may cause a user interface 306, depicted on FIG. 4, to be presented. In particular, the user has selected Emily in the user interface 304. From the user interface 306, the user can turn on “Unicorn” for Emily by selecting turn on button 328 (e.g., create a new restricted mobile payment account). Following selection of the turn on button 328, the unrestricted user device 106 may present user interface 308. The user interface 308 may present details about the new account, warnings, a privacy statement, and any other permissions and/or require clickthrough. In some examples, the user interface 308 may include an option to review and accept certain terms relating to the trusted family circle 114 and payment accounts. If the user desires to continue, she may select continue button 330.

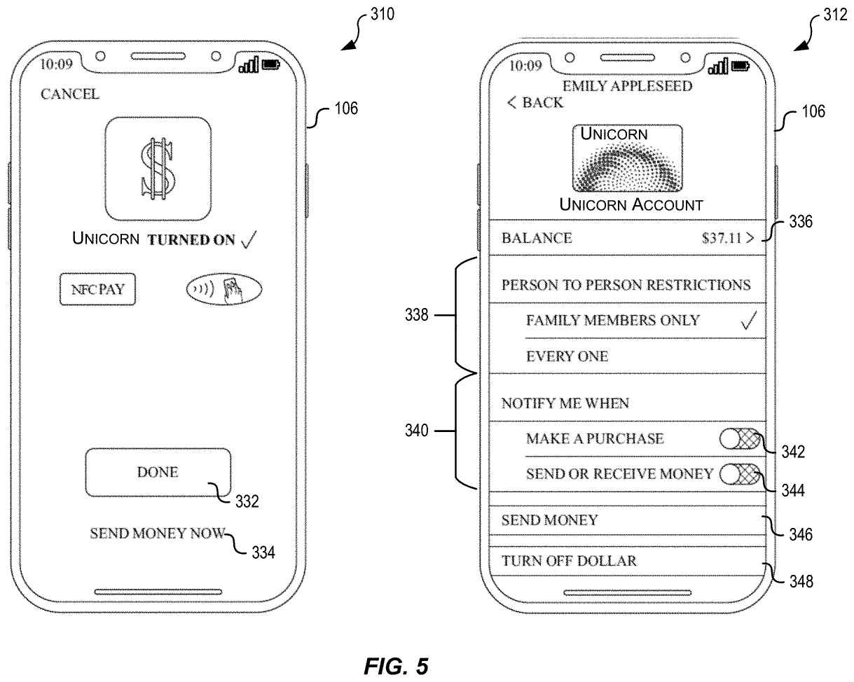

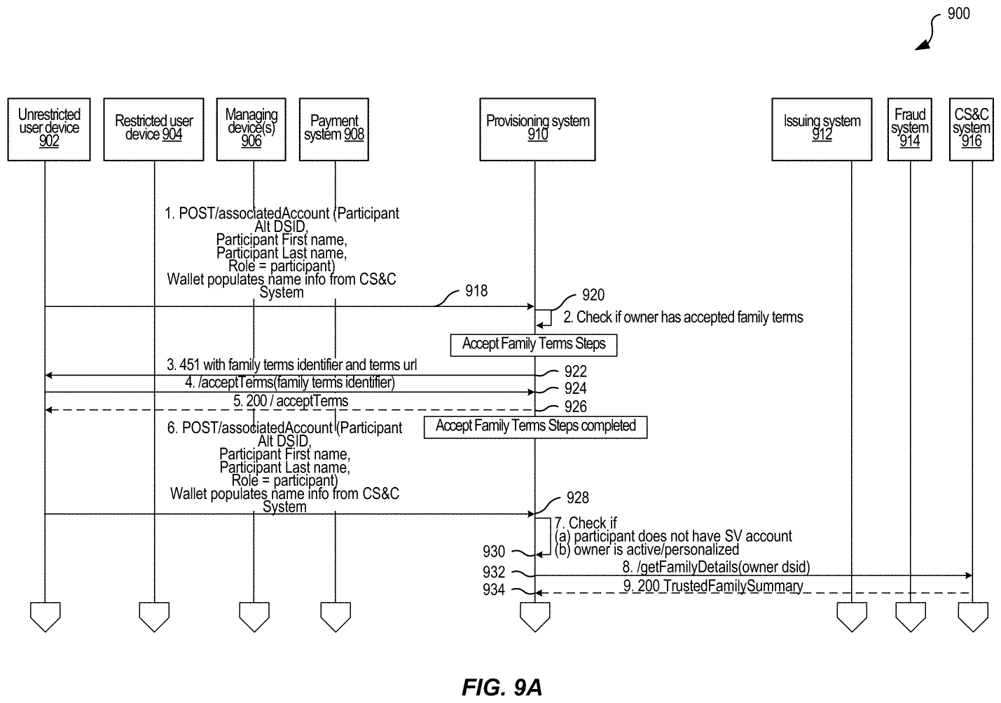

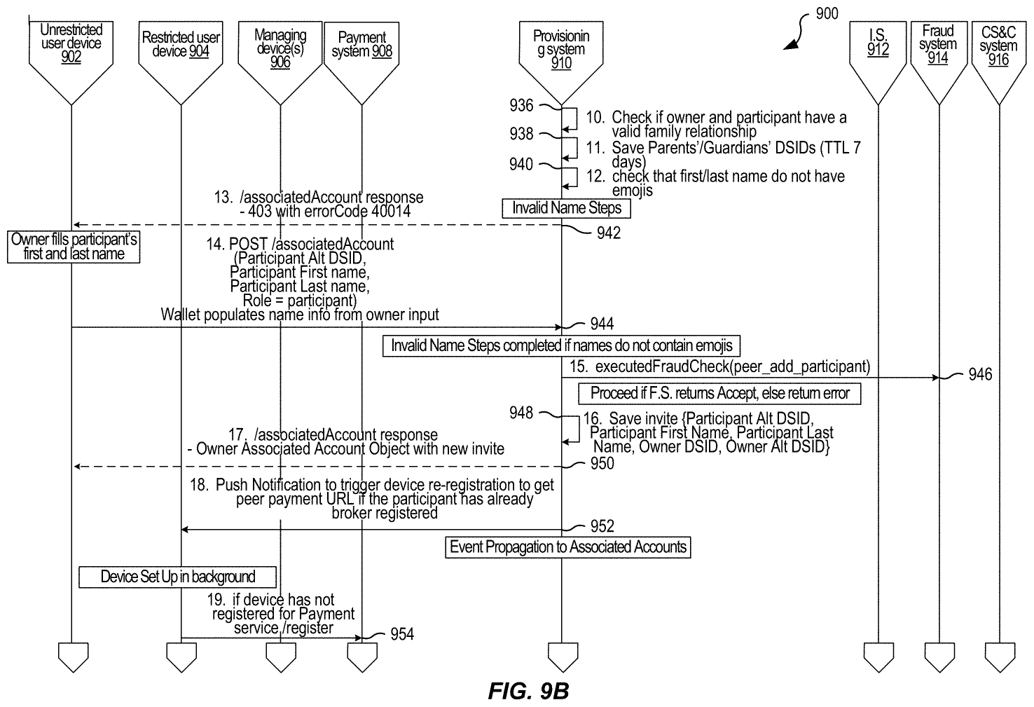

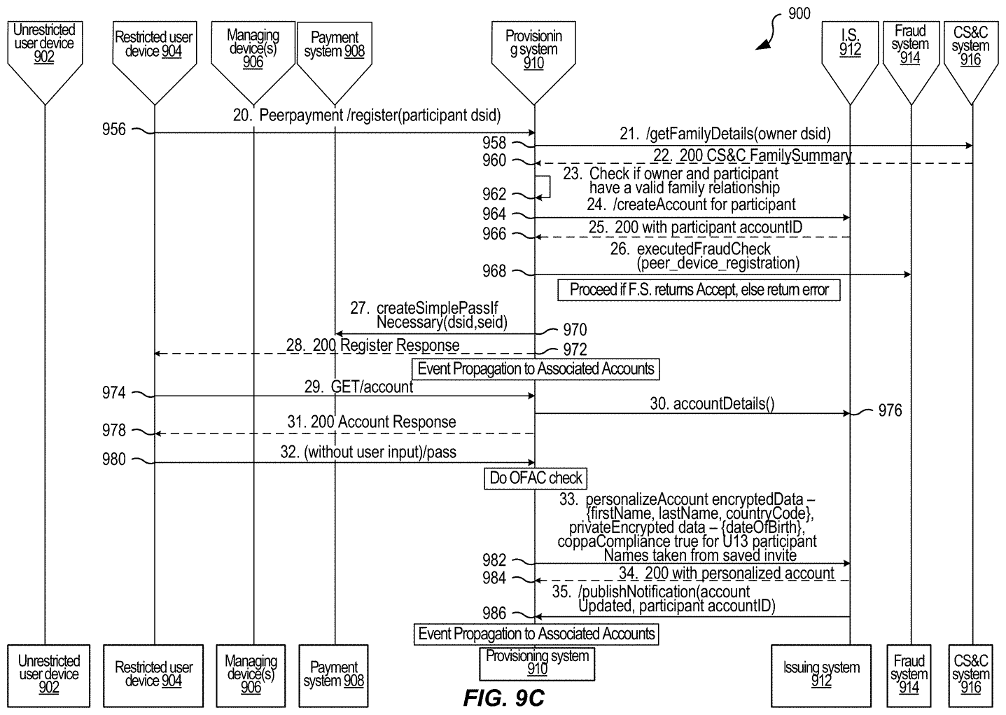

Selection of the continue button 330, may cause the unrestricted user device 106 to present a user interface 310, as depicted in FIG. 5. The user interface 310 constitutes a confirmation screen, confirming that the restricted mobile payment account has been created for Emily. In the background, as described with respect to FIGS. 9-11, other communications, checks, and verifications have been conducted to ensure that Emily can use the account to make payments and receive money. The user interface 310 includes a done button 332 and a send money now button 334. Selection of the done button 332 may cause the application to close. Selection of the send money now button 334 may enable Jane to share money directly with Emily (or other users) using the new account. To do so, the unrestricted user device 106 may open a messaging application through which the peer-to-peer transaction (e.g., Jane to Emily) may be sent.

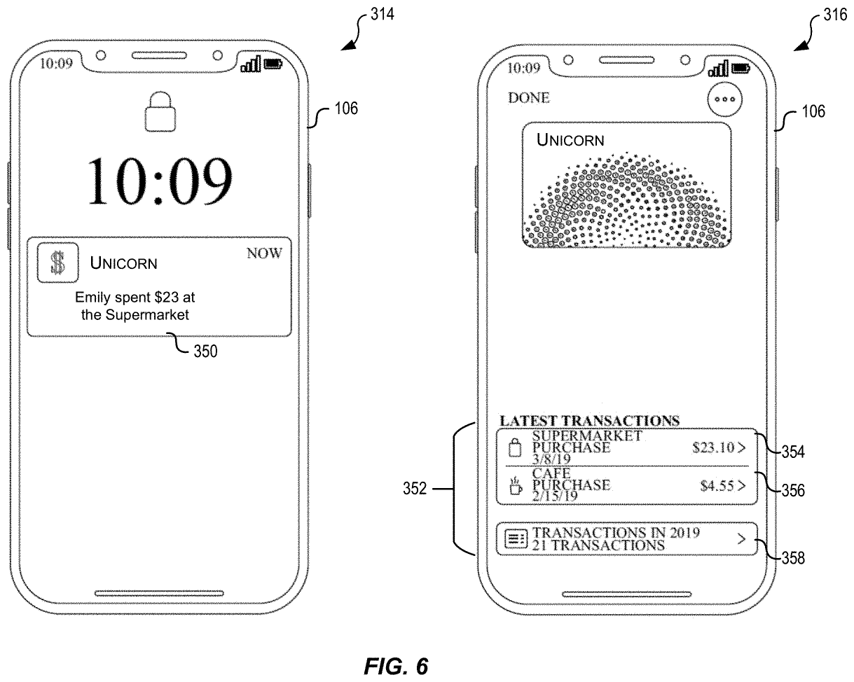

As depicted in FIG. 5, user interface 312 depicts a management user interface by which Jane (e.g., owner user) can manage aspects of Emily's restricted mobile payment account. In particular, the user interface 312 includes a balance indicator 336, a person restrictions area 338, a notifications area 340 that includes a purchase notification toggle 342 and a send/receive money toggle 344, a send money button 346, and a turn off button 348. The balance indicator 336 identifies the current balance in the restricted account. The person restrictions area 338 identifies which, if any, restrictions Jane has set of Emily. In this case, the restriction is set to family members only, meaning Emily can only send and receive money from those in the trusted family circle 114. In some cases other restrictions may be set at different levels of granularity. For example, a restrictions may indicate that Emily can only send and receive money from those in Emily's contacts, those nearby Emily (e.g., within the same geographic proximity), those who are Emily's social media friends or followers, those who are on a predefined list (e.g., a list of approved accounts outside that family and which is maintained by the Jane or other account owner), and any other suitable restriction. The notifications area 340 identifies the current notification settings, which in this case, include Jane getting notifications of new purchases (e.g., the toggle 342 is set to on) and when money is sent and received (e.g., the toggle 344 is set to on). Thus, Jane will receive notifications on her user device whenever any of these transactions occur using Emily's restricted mobile payment account. For example, user interface 314, as depicted in FIG. 6, includes an example transaction activity notification 350. The notification 350 indicates that Emily spent $23 at the Supermarket. A record of this transaction may also be saved in a transaction table. Information from the transaction table may be depicted in a transaction area 352 in a user interface 316. The transaction area 352 includes recent transactions 354 and 356, and a list of all transactions for 2019. The transaction 354 corresponds to the notification 350. The transaction area 352 also includes an option 358 to view historical transactions (e.g., for the year 2019).

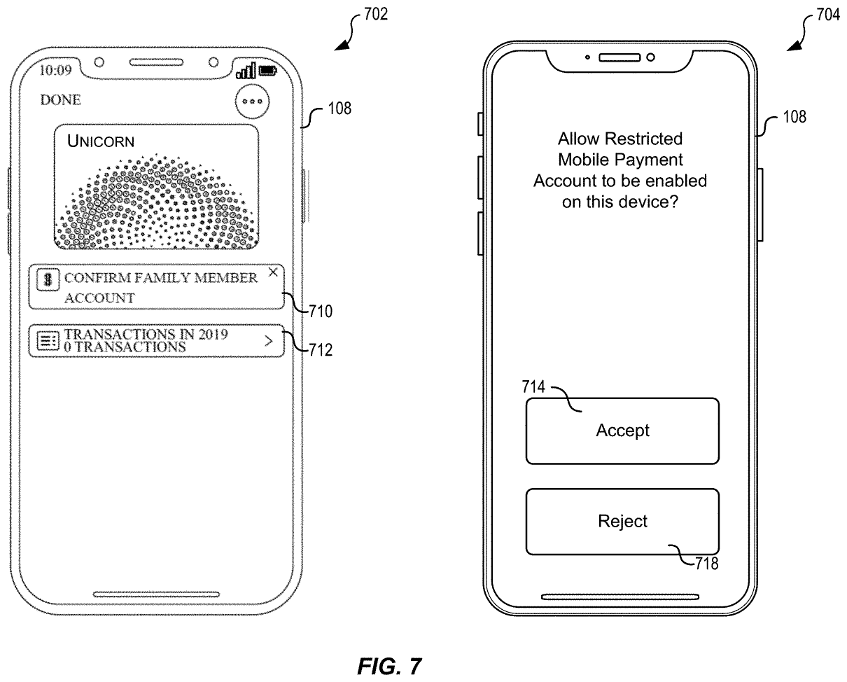

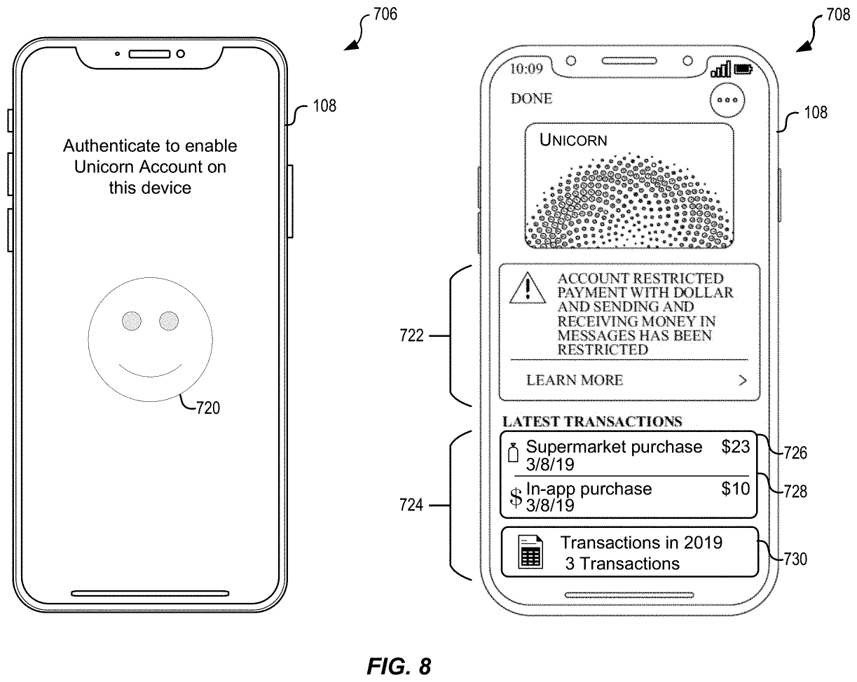

FIGS. 7 and 8 illustrate user interfaces for establishing restricted mobile payment accounts for use on user devices, according to various examples. The user interfaces in FIGS. 7 and 8 in particular may be presented on the restricted user device 108 and may represent the views presented to a user as part of new restricted account creation and management.

FIGS. 7-8 illustrate user interfaces 702-708 presented on the restricted user device 108. In particular, the user interface 702 may be presented responsive to an owner user requesting establishment of a restricted mobile payment account on behalf of the user of the restricted user device 108. Thus, the user interface 702 includes a confirmation option 710 and a transaction history area 712. The transaction history area 712 identifies zero transactions because the account has not yet been established. The confirmation option 710, when selected, causes the restricted user device 108 to conduct a workflow to establish the payment account. In some examples, the owner user may have previously requested creation of the payment account, and the restricted user may need to go through the workflow to finalize creation of the account.

Selection of the confirmation option 710, also causes the restricted user device 108 to present a user interface 704. The user interface 704 includes a prompt about whether to allow creation of a restricted mobile payment account (e.g., a Unicorn Account) on the user device 108. The user interface 704 also includes options to accept 714 or reject 718 the creation of the restricted mobile payment account on the restricted user device 108. Selection of a button corresponding to the reject 718 cancels the creation of the account. Selection of the accept button 714 causes the restricted user device 108 to present user interface 706, as depicted in FIG. 8. In the user interface 708, the user is presented with an option to authenticate their account. This may be performed using a facial scan, as depicted by face image 720. In some examples, the user may input her login credentials, which may be the same credentials (or at least linked with) the credentials stored in the trusted family circle 114. Once the account has been created, a user interface 708 may be presented on the restricted user device 108. The user interface 708 includes a restriction area 722 and a transaction area 724. A record of the transaction shown in the transaction area 724 may be saved in a transaction table. Information from the transaction table may be depicted the transaction area 724, which includes recent transactions 726 and 728, and a list 730 of all transactions for 2019. The transaction 726 corresponds to the transaction 354 and the notification 350, previously discussed as being presented at the unrestricted user device 106. The restriction area 722 indicates the restrictions on the restricted mobile payment account, which correspond to the information presented in the person restrictions area 338 and the notifications area 340.

FIGS. 9A-9C illustrate a sequence diagram 900 showing an example process for establishing restricted mobile payment accounts for use on user devices, according to various examples. In particular, the sequence diagram 900 depicts processes for creating a new restricted mobile payment account, such as the process described with respect to FIGS. 3-6.

As elements in the sequence diagram 900, FIGS. 9A-9C include the unrestricted user device 902 (e.g., the unrestricted user device 106), a restricted user device 904 (e.g., the restricted user device 108), a managing user device 906, a payment system 908 (e.g., the payment system 250), a provisioning system 910 (e.g., the provisioning system 204), an issuing system 912 (e.g., the issuing system 220), a fraud system 914 (e.g., the fraud verification system 280), and the cloud storage and compute system 916 (e.g., the CS&C system 206). The function described with respect to the unrestricted user device 902 and the restricted user device 904 may be performed by and/or within digital wallet applications, within the operating system, or within other applications.

Beginning with FIG. 9A, at 918, the unrestricted user device 902 requests the provisioning system 910 to create a new restricted mobile payment account (e.g., an associated account). In some examples, this request may be referred to as an “invite.” The request includes user account information associated with the restricted user (e.g., participant). This information may be prepopulated at the unrestricted user device 902 from the CS&C system 916 and/or input by the user. At 920, the provisioning system 910 checks to see if the unrestricted user (e.g., the owner) has accepted certain terms relating to the trusted family circle. If not, at 922, the provisioning system 910 sends a message to the unrestricted user device 902 that includes a URL for the user to accept the terms. In response, at 924, the unrestricted user device 902 sends an acceptance to the provisioning system 910, and an acknowledgement is sent at 926. At 928, the unrestricted user device 902 sends the provisioning system 910 another similar request as at 918. In some examples, block 928 may be omitted, e.g., if the user had already accepted the family terms as checked at 920. At 930, the provisioning system 910 determines whether the restricted user already has a payment account and whether the owner is active. At 932, the provisioning system 910 requests details, from the CS&C system 916, about the trusted family circle using a unique account identifier associated with the account owner from the CS&C system 916. The CS&C system 916 may generally be configured to maintain details about the trusted family circle. In response, at 934, the CS&C system 916 returns to the provisioning system 910 a summary of the trusted family.

Turning now to FIG. 9B, at 936, the provisioning system 910 checks if the owner and participant have a valid family relationship. At 938, the provisioning system 910 saves information relating to other adults in the trusted family circle. At 940, the provisioning system 910 performs a name check on the user account name of the restricted user to confirm that the name meets certain requirements, e.g., does not include emoji's. If one or both of the first name and last name are invalid, at 942, the provisioning system 910 sends an error code to the unrestricted user device 902. The owner user may input the correct first and last name and, at 944, the unrestricted user device 902 may send a request to the provisioning system 910 to create the restricted mobile payment account. In this example, the owner user inputs the name information, rather than it being populating automatically from the CS&C system 916. At 946, the provisioning system 910 may communicate with the fraud system 914 to perform a fraud check, which may include providing at least some user account information to the fraud system 914. At 948, the provisioning system 910 saves at least some portion of the invite received previously from the unrestricted user device 902. This may include unique identifiers for the participant and owner, along with alternate identifiers, and first and last names of the participant. At 950, the provisioning system 910 sends a response to the unrestricted user device 902 that includes an account object. At 952, the provisioning system 910 sends a push notification to the restricted user device 904 to cause the restricted user device 904 to begin a workflow for registering to get payment information (e.g., a payment URL and a token). The workflow may be performed in the background on the restricted user device 904. In some examples, it may include, at 954, the restricted user device 904 registering with the payment system 908.

Turning now to FIG. 9C, at 956, the restricted user device 904 may register for peer-to-peer payment with the provisioning system 910. This may include sharing a unique user account identifier with the provisioning system 910. At 958, the provisioning system 910 requests the trusted family circle details from the CS&C system 916. In response, at 960, the CS&C system 916 returns a summary about the trusted family circle. At 962, the provisioning system 910 checks if the owner and participant have a valid family relationship, like at 936. At 964, the provisioning system 910 causes creation of the restricted mobile payment account by communicating with the issuing system 912. In response, at 966, the issuing system 912 acknowledges and returns with the participant account identifier. The participant account ID may represent an identifier that uniquely identifies the restricted mobile payment account. At 968, the provisioning system 910 executes a fraud check by communicating with the fraud detection system 914. At 970, the provisioning system 910 may create a payment processing pass with the payment system 908. At 972, the provisioning system 910 sends a registered response to the restricted user device 904. At 974, the restricted user device 904 requests account details relating to the mobile payment account. At 976, the provisioning system 910 requests the account details from the issuing system 912. At 978, the provisioning system 910 sends an acknowledgement to the restricted user device 904. At 980, the restricted user device 904 responds to the provisioning system 910 with the pass. At 982, the provisioning system 910 performs a workflow relating to an Office of Foreign Assets Control (OFAC) check. This may include, at 982, the provisioning system 910 providing mobile payment account data to the issuing system 912. At 984, the issuing system 912 sends an acknowledgement with the personalized account information. At 986, the issuing system 912 may send a communication to the provisioning system 910 that includes the updated account information.

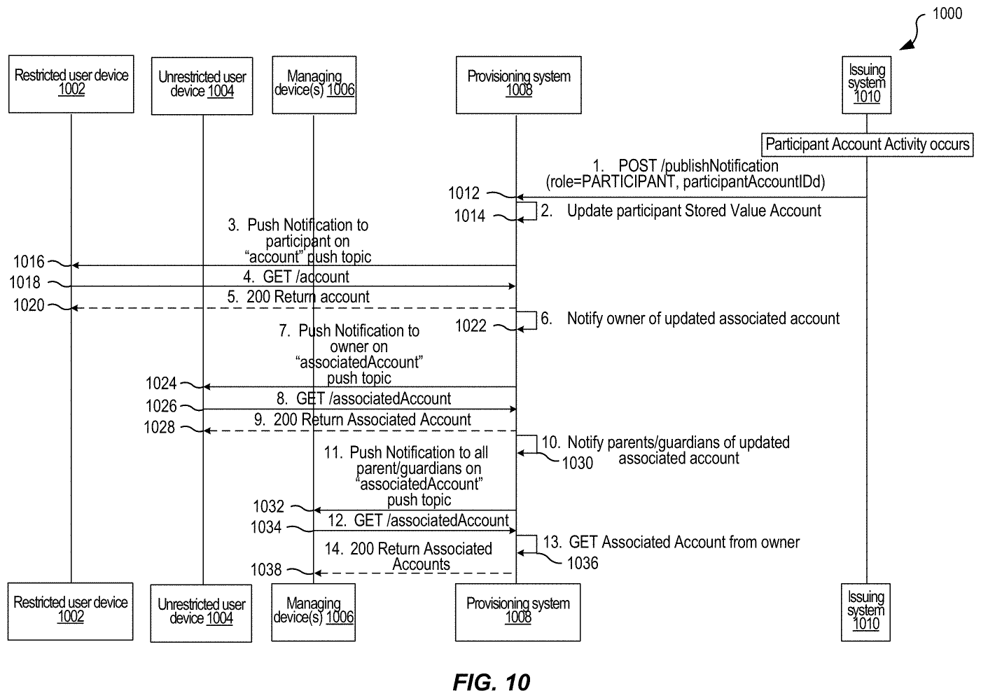

FIG. 10 illustrates a sequence diagram 1000 showing an example process for establishing restricted mobile payment accounts for use on user devices, according to at least one example. In particular, the sequence diagram 1000 depicts processes for registering and sharing activity transaction notifications.

As elements in the sequence diagram 1000, FIG. 10 includes a restricted user device 1002 (e.g., the restricted user device 108), an unrestricted user device 1004 (e.g., the unrestricted user device 106), a managing user device 1006, a provisioning system 1008 (e.g., the provisioning system 204), and an issuing system 1010 (e.g., the issuing system 220). The function described with respect to the unrestricted user device 902 and the restricted user device 904 may be performed by and/or within digital wallet applications, within the operating system, or within other applications.

At 1012, the issuing system 1010 informs the provisioning system 1008 about new activity on a restricted mobile payment account. At 1014, the provisioning system 1008 updates a stored value account associated with the restricted mobile payment account. This may include updating a table of the transactions as maintained by the provisioning system 1008. At 1016, the provisioning system 1008 sends a push notification to the restricted user device 1002 to obtain information about the recent transaction. In response, at 1018, the restricted user device 1002 returns information about the recent transaction. At 1020, the provisioning system 1008 acknowledges the message. At 1022, the provisioning system 1008 notifies the owner of the updates to the restricted mobile payment account (e.g., the associated account). At 1024, the provisioning system 1008 sends a push notification to the unrestricted user device 1004 on the associated account topic. In response, at 1026, the unrestricted user device 1004 requests information about the restricted mobile payment account. At 1028, the provisioning system 1008 sends an acknowledgement including the updates. At 1030, the provisioning system 1008 notifies the other users of the updates. This may include, at 1032, sending a push notification to the managing device(s) 1006, receiving responses at 1034, and returning the updates, at 1038.

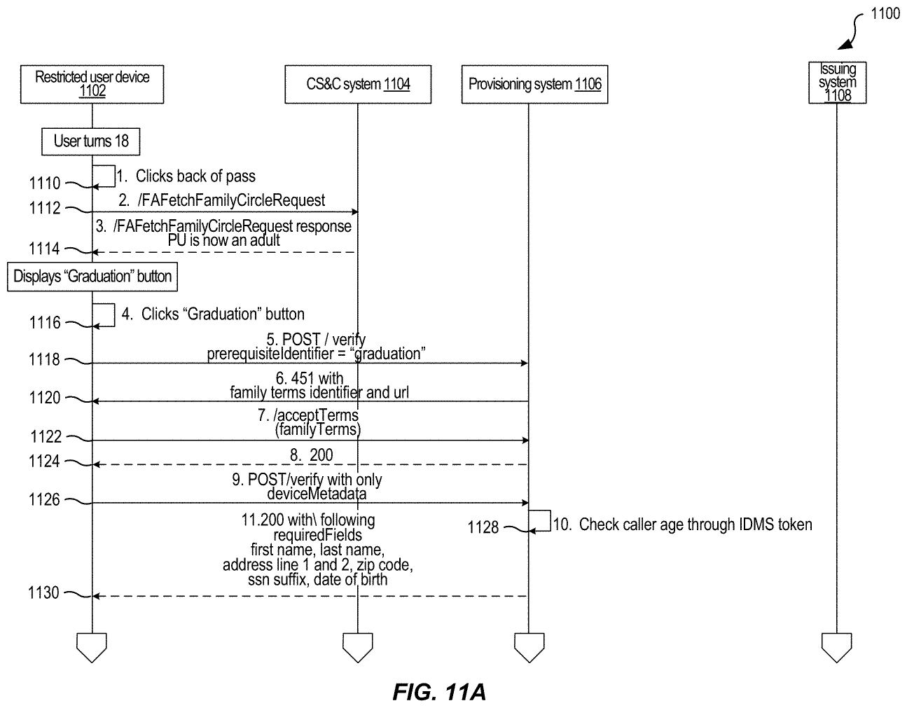

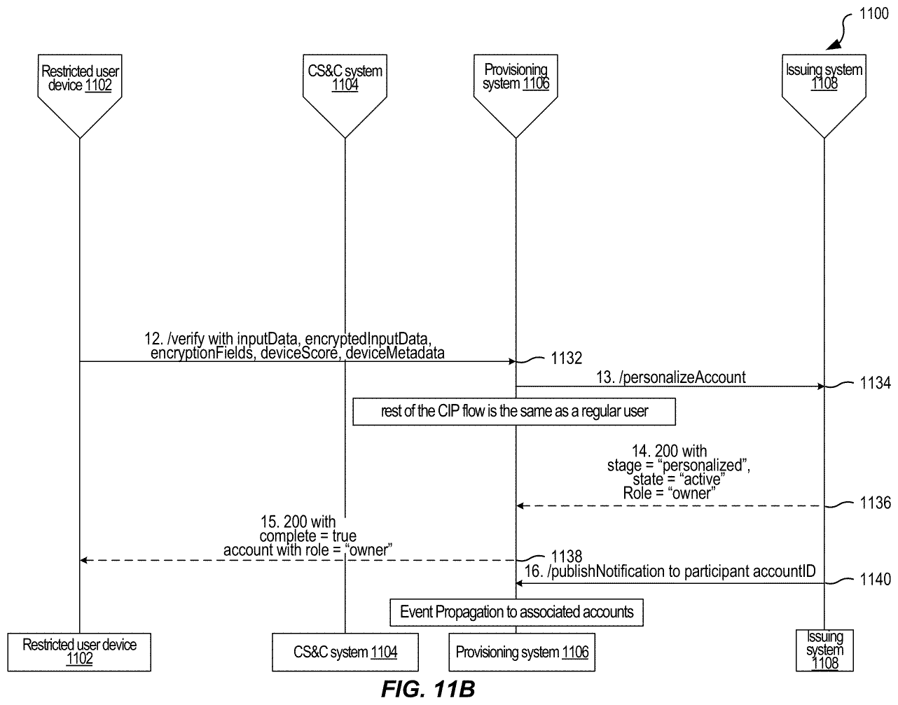

FIGS. 11A and 11B illustrate a sequence diagram 1100 showing an example process for converting a restricted mobile payment account to a primary account, according to at least one example. In particular, the sequence diagram 1100 depicts a process for converting a restricted mobile payment account created, as described herein, to a primary account, e.g., when the restricted user turns 18 years old.

As elements in the sequence diagram 1100, FIG. 11A and 11B include a restricted user device 1102 (e.g., the restricted user device 108), a CS&C system 1104 (e.g., a CS&C system 206), a provisioning system 1106 (e.g., the provisioning system 204), and an issuing system 1108 (e.g., the issuing system 220). The function described with respect to the restricted user device 1102 may be performed by and/or within a digital wallet application, within the operating system, or within other applications.

Beginning with FIG. 11, at 1110, after the user of the restricted user device 1102 turns 18 (or any other suitable age or when any other suitable event occurs that results in the user's status changing), the user clicks a back of a digital pass that represents the restricted mobile payment account. At 1112, the restricted user device 1102 requests information about the trusted family circle from the CS&C system 1104. In response, the CS&C system 1104, at 1114 sends information to the restricted user device 1102. At this point, the restricted user device 1102 displays a graduation button. At 1116, the restricted user on the restricted user device 1102 clicks the graduation button. This may begin the workflow for converting the user's account. At 1118, the restricted user device 1102 sends an message to the provisioning system 1106. At 1120, the provisioning system 1106 returns with a family identifier and uniform resource locator. At 1122, the restricted user device 1102 accepts the family terms. At 1124, the provisioning system 1106 sends an acknowledgement. At 1126, the restricted user device 1102 sends device metadata to the provisioning system 1106. At 1128, the provisioning system 1106 checks the age of the user using a token. At 1130, the provisioning system 1106 sends information to the restricted user device 1102.

Turning now to FIG. 11B, at 1132, the restricted user device 1102 sends a verification to the provisioning system 1106. At 1134, the provisioning system 1106 sends a request to the issuing system 1108 to personalize the account. The remaining steps of 1136, 1138, and 1140 proceed similarly as described previously.

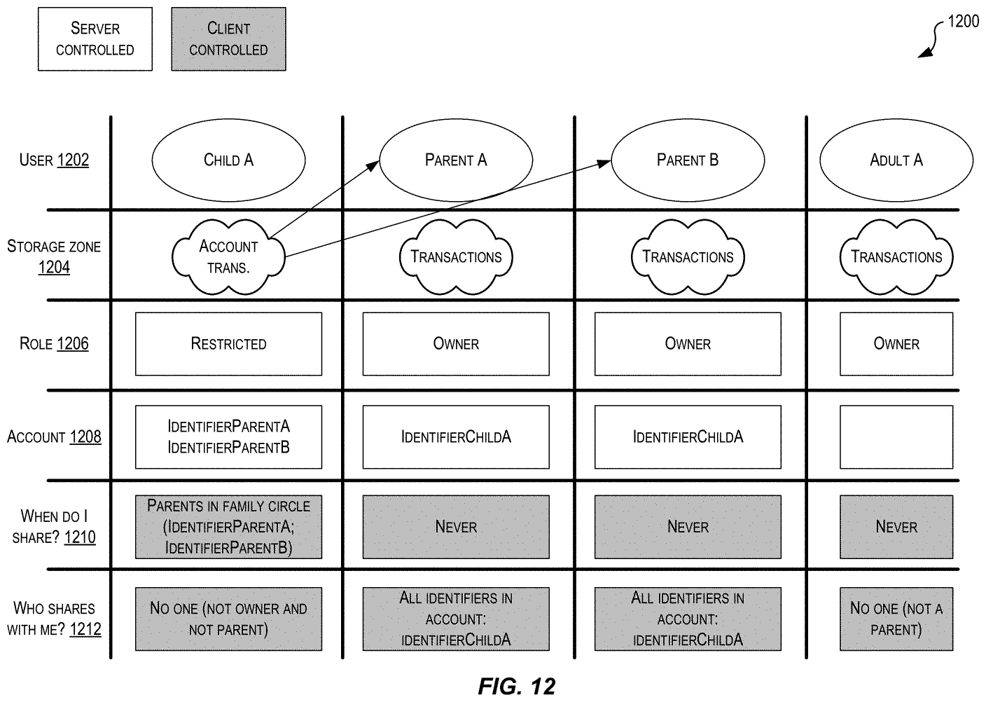

FIG. 12 illustrates a table 1200 showing an example process for sharing data with and among mobile payment accounts, according to at least one example. The table 1200 identifies the users 1202, storage zones 1204 (e.g., table of activity transaction records), roles assigned to the users 1206, account details 1208, outbound sharing rules 1210, and inbound sharing rules 1212. The items in rectangle boxes in white are controlled by the server (e.g., the service provider 104). Boxes in gray are controlled by the client device (e.g., one of the user devices 106, 108). The users 1202 are identified as a child A, a parent A, a parent B, and an adult A. The roles assigned to the users 1206 may be that of restricted, owner, and/or managing participant. In some examples, the adult A is a managing participant but may also be identified as an owner for purposes of this table. The account 1208 indicate which accounts are associated with which other accounts. Thus, the child's account is associated with both parents A and B. The parents' accounts are each associated with the child A. The outbound sharing rules 1210 indicate whether the accounts share their own transaction information with others in the trusted family circle. The child A shares with both parents A and B, as illustrated by the directional arrows. The inbound sharing rules 1212 indicate whether the accounts receive information. The child A does not receive any sharing. Both parents A and B receive sharing from the child A.

The storage zones 1204 are cloud-based storage locations (e.g., on the CS&C system 206) where transactions are stored. Each storage zone 1204 is encrypted using an encryption key.

The data may be decrypted by a device that has the corresponding key. When a restricted mobile payment accounts is established, all users with the owner role will get the keys and a URL to access the storage zone of the child A. This enables the devices of the other users to request and receive transaction activity data from the storage zone. When a child graduates (e.g., their account is converted to a primary account), the keys may be revoked such that the other users can no longer access the storage zone of the child A. The other users' devices will also delete their keys and the existing data cached on those devices.

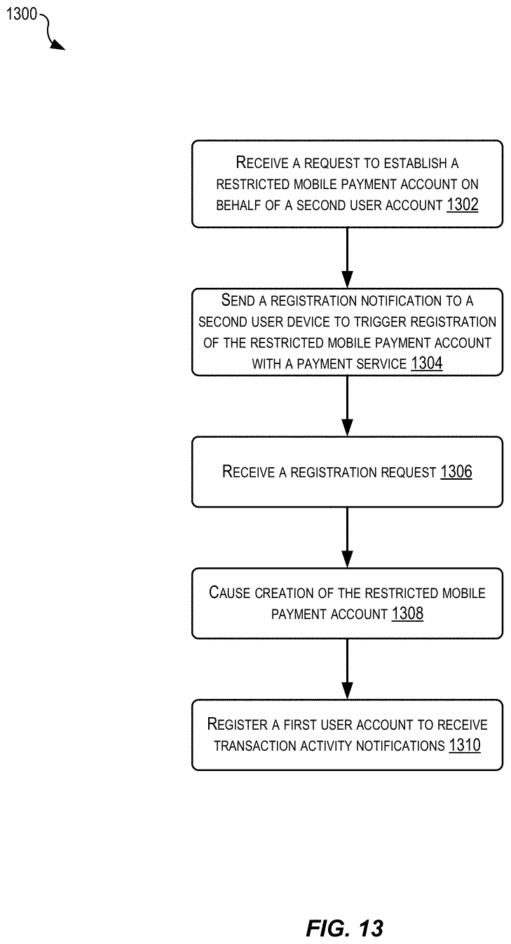

FIG. 13 illustrates a flowchart showing an example process 1300 for establishing restricted mobile payment accounts for use on user devices, according to at least one example. The process 1300 may be performed by the service provider 104 and in particular the provisioning system 204 of the service provider 104. The process 1300 may relate to establishing a new account, for example, as described with reference to FIGS. 3-6 and 9A-9C. For example, using the process 1300, a user on a first device may cause creation of a restricted mobile payment account on a second device. The second device may have shared account information with the first device or may be separate.

The process 1300 begins at 1302 by the provisioning system 204 (FIG. 2) receiving a request to establish a restricted mobile payment account on behalf of a second user account. The request may be received from a first user device (e.g., an unrestricted user device) associated with a first user account. The restricted mobile payment account will be a subaccount of a primary mobile payment account of the first user account. In some examples, the request may include user account information that is associated with the second user account. In some examples, the user account information may be obtained from a remote storage and compute system or input at the first user device.

In some examples, the process 1300 may further include determining that the second user account is included in a user account group (e.g., a user account family) with the first user account based at least in part on the user account information. In some examples, the user account group may include an owner role and at least one of a participant role or a managing participant role. In some examples, the first user account is the owner role and the second user account is the participant role.

At 1304, the process 1300 includes the provisioning system 204 sending a registration notification to a second user device to trigger registration of the restricted mobile payment account with a payment service (e.g., the provisioning system 204). The second user device may be associated with the second user account. The payment service may be a peer-to-peer payment service.

At 1306, the process 1300 includes the provisioning system 204 receiving a registration request that includes a unique user account identifier of the second user account. The registration request may be received from the second user device and based at least in part on the registration notification.

At 1308, the process 1300 includes the provisioning system 204 causing creation of the restricted mobile payment account. This may be based at least in part on the user account information and the unique user account identifier of the second user account. In some examples, this may include sending an account creation request to an external issuing system, and receiving a confirmation of execution of the account creation request. The confirmation may include a payment account identifier of the restricted mobile payment account.

At 1310, the process 1300 includes registering the first user account to receive transaction activity notifications. The transaction activity notifications may correspond to transactions of the restricted mobile payment account. In some examples, registering the first user account to receive the transaction activity notifications may include sharing a token (e.g., a cryptographic key) and a resource locator (e.g., a URL to the zone) with the first user device. The resource locator may identifier a network location (e.g., storage zone) of a transaction activity table that stores the transactions of the restricted mobile payment account. The token may be usable to access the transaction activity table.

In some examples, the process 1300 further includes the provisioning system 204 receiving, from an issuing system, an indication of a new transaction of the restricted mobile payment account. The process 1300 may further include sending, to the first user device and based at least in part on the indication, a transaction activity notification corresponding to the new transaction. The process 1300 may further include receiving, from the first user device, a request for transaction information corresponding to the new transaction. The process 1300 may further include sending, to the first user device and based at least in part on the request, the transaction information. In this example, the request for transaction information may include a token and a resource locator. In which case, the process 1300 may further include the provisioning system 204 accessing a transaction activity table at a resource location identified by the resource locator, and using the token to read the transaction information in the transaction activity table.

In some examples, the process 1300 may further include the provisioning system 204 registering a third user account to receive the transaction activity notifications, the third user account may be in the user account group. In some examples, the process 1300 may further include storing the user account information included in the request to establish the restricted mobile payment account on behalf of the second user account. In some examples, the process 1300 may further include the provisioning system 204 communicating with an account verification system to verify the second user account based at least in part on the user account information.

In some examples, the process 1300 may further include the provisioning system 204 converting the restricted mobile payment account to a new primary mobile payment account of the second user account based at least in part on detecting that an age associated with the second user account meets or exceeds an age threshold (e.g., 18 years of age—when the user becomes an adult). In some example, the age threshold may be more than 18 or less than 18. In some examples, a different threshold or rule may be used. For example, rather than detecting based on age, the detecting may be based on change to a relationship status, legal status, and the like. In some examples, the process 1300 may further include the provisioning system 204, prior to converting the restricted mobile payment account: presenting a conversion option at a user interface of the second user device based at least in part on detecting that the age associated with the second user account meets or exceeds the age threshold, and responsive to a selection of the conversion option at the user interface, verifying an identity of a user of the second user account. In this example, converting the restricted mobile payment account may be based at least in part on verifying the identity of the user of the second user account.

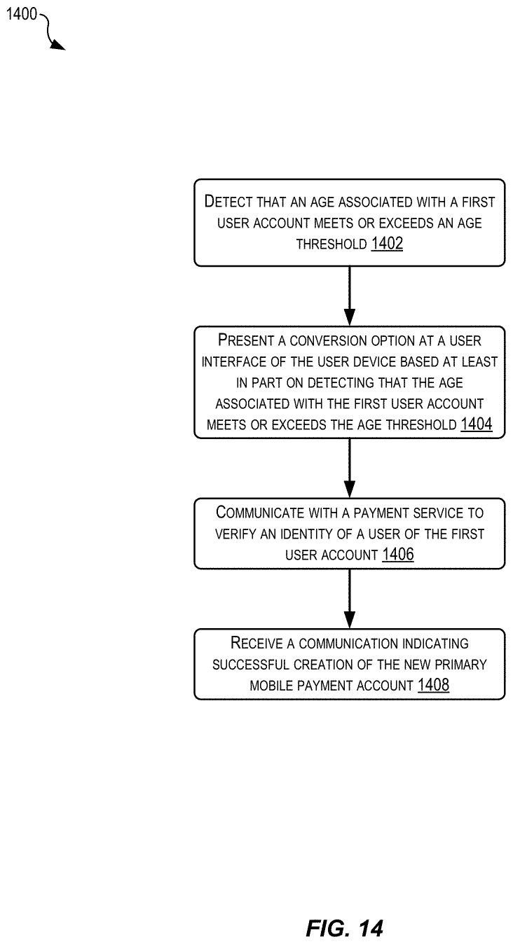

FIG. 14 illustrates a flowchart showing an example process 1400 for converting a restricted mobile payment account, according to at least one example. The process 1400 may be performed by the restricted user device 108. The process 1400 may relate to converting a restricted mobile payment account to a primary account as described, for example, with reference to FIGS. 11A and 11B.

The process 1400 begins at 1402 by the restricted user device 108 (FIG. 1) detecting that an age associated with a first user account meets or exceeds an age threshold. The first user account may be associated with a restricted mobile payment account that is a subaccount of a primary account of a second user account.

At 1404, the process 1400 may include the restricted user device 108 presenting a conversion option at a user interface of the user device based at least in part on detecting that the age associated with the first user account meets or exceeds the age threshold. The conversion option may enable conversion of the restricted mobile payment account to a new primary mobile payment account of the first user account.

At 1406, the process 1400 may include the restricted user device 108, responsive to a selection of the conversion option at the user interface, communicate with a payment service to verify an identity of a user of the first user account. In some examples, communicating with payment service may include providing device metadata to the payment service to verify the age, and responsive to a request from the payment service, providing user account information to the payment service to verify additional user account details.

At 1408, the process 1400 may include the restricted user device 108 receiving a communication indicating successful creation of the new primary mobile payment account.

In some examples, the process 1400 may further include the restricted user device 108 receiving a transaction activity notification corresponding to a new transaction of the new primary mobile payment account, sending a request for transaction information corresponding to the new transaction, and receiving, based at least in part on the request, the transaction information.

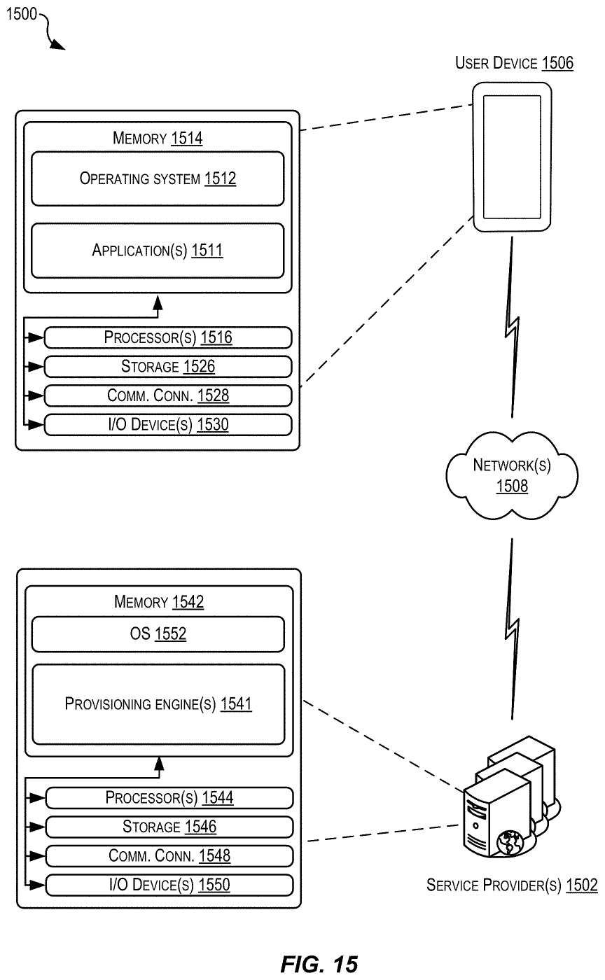

FIG. 15 illustrates an example architecture or environment 1500 configured to implement techniques described herein, according to at least one example. In some examples, the example architecture 1500 may further be configured to enable a user device 1506 and service provider computer 1502 to share information. The service provider computer 1502 is an example of the service provider 104, the issuing system 220, and the issuing system 220, and a fraud verification system 280. The user device 1506 is an example of the user devices 106 and 108. In some examples, the devices may be connected via one or more networks 1508 (e.g., via Bluetooth, WiFi, the Internet, or the like). In some examples, the service provider computer 1502 may be configured to implement at least some of the techniques described herein with reference to the user device 1506.

In some examples, the networks 1508 may include any one or a combination of many different types of networks, such as cable networks, the Internet, wireless networks, cellular networks, satellite networks, other private and/or public networks, or any combination thereof. While the illustrated example represents the user device 1506 accessing the service provider computer 1502 via the networks 1508, the described techniques may equally apply in instances where the user device 1506 interacts with the service provider computer 1502 over a landline phone, via a kiosk, or in any other manner. It is also noted that the described techniques may apply in other client/server arrangements (e.g., set-top boxes, etc.), as well as in non-client/server arrangements (e.g., locally stored applications, peer-to-peer configurations, etc.).

As noted above, the user device 1506 may be any type of computing device such as, but not limited to, a mobile phone, a smartphone, a personal digital assistant (PDA), a laptop computer, a desktop computer, a thin-client device, a tablet computer, a wearable device such as a smart watch, or the like. In some examples, the user device 1506 may be in communication with the service provider computer 1502 via the network 1508, or via other network connections.

In one illustrative configuration, the user device 1506 may include at least one memory 1514 and one or more processing units (or processor(s)) 1516. The processor(s) 1516 may be implemented as appropriate in hardware, computer-executable instructions, firmware, or combinations thereof. Computer-executable instruction or firmware implementations of the processor(s) 1516 may include computer-executable or machine-executable instructions written in any suitable programming language to perform the various functions described. The user device 1506 may also include geo-location devices (e.g., a global positioning system (GPS) device or the like) for providing and/or recording geographic location information associated with the user device 1506.

The memory 1514 may store program instructions that are loadable and executable on the processor(s) 1516, as well as data generated during the execution of these programs. Depending on the configuration and type of the user device 1506, the memory 1514 may be volatile (such as random access memory (RAM)) and/or non-volatile (such as read-only memory (ROM), flash memory, etc.). The user device 1506 may also include additional removable storage and/or non-removable storage 1526 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 1514 may include multiple different types of memory, such as static random access memory (SRAM), dynamic random access memory (DRAM), or ROM. While the volatile memory described herein may be referred to as RAM, any volatile memory that would not maintain data stored therein once unplugged from a host and/or power would be appropriate.

The memory 1514 and the additional storage 1526, both removable and non-removable, are all examples of non-transitory computer-readable storage media. For example, non-transitory computer readable storage media may include volatile or non-volatile, removable or non-removable media implemented in any method or technology for storage of information such as computer-readable instructions, data structures, program modules, or other data. The memory 1514 and the additional storage 1526 are both examples of non-transitory computer storage media. Additional types of computer storage media that may be present in the user device 1506 may include, but are not limited to, phase-change RAM (PRAM), SRAM, DRAM, RAM, ROM, Electrically Erasable Programmable Read-Only Memory (EEPROM), flash memory or other memory technology, compact disc read-only memory (CD-ROM), digital video disc (DVD) or other optical storage, magnetic cassettes, magnetic tape, magnetic disk storage or other magnetic storage devices, or any other medium that can be used to store the desired information and that can be accessed by the user device 1506. Combinations of any of the above should also be included within the scope of non-transitory computer-readable storage media. Alternatively, computer-readable communication media may include computer-readable instructions, program modules, or other data transmitted within a data signal, such as a carrier wave, or other transmission. However, as used herein, computer-readable storage media does not include computer-readable communication media.

The user device 1506 may also contain communications connection(s) 1528 that allow the user device 1506 to communicate with a data store, another computing device or server, user terminals, and/or other devices via the network 1508. The user device 1506 may also include I/O device(s) 1530, such as a keyboard, a mouse, a pen, a voice input device, a touch screen input device, a display, speakers, a printer, etc.

Turning to the contents of the memory 1514 in more detail, the memory 1514 may include an operating system 1512 and/or one or more application programs or services for implementing the features disclosed herein such as applications 1511 (e.g., digital wallet, settings application, third-party applications, browser application, etc.). In some examples, the service provider computer 1502 may also include an application to perform similar techniques as described with reference to the user device 1506. Similarly, at least some techniques described with reference to the service provider computer 1502 may be performed by the user device 1506.

The service provider computer 1502 may also be any type of computing device such as, but not limited to, a collection of virtual or “cloud” computing resources, a remote server, a mobile phone, a smartphone, a PDA, a laptop computer, a desktop computer, a thin-client device, a tablet computer, a wearable device, a server computer, a virtual machine instance, etc. In some examples, the service provider computer 1502 may be in communication with the user device 1506 via the network 1508, or via other network connections.

In one illustrative configuration, the service provider computer 1502 may include at least one memory 1542 and one or more processing units (or processor(s)) 1544. The processor(s) 1544 may be implemented as appropriate in hardware, computer-executable instructions, firmware, or combinations thereof. Computer-executable instruction or firmware implementations of the processor(s) 1544 may include computer-executable or machine-executable instructions written in any suitable programming language to perform the various functions described.

The memory 1542 may store program instructions that are loadable and executable on the processor(s) 1544, as well as data generated during the execution of these programs. Depending on the configuration and type of service provider computer 1502, the memory 1542 may be volatile (such as RAM) and/or non-volatile (such as ROM, flash memory, etc.). The service provider computer 1502 may also include additional removable storage and/or non-removable storage 1546 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 1542 may include multiple different types of memory, such as SRAM, DRAM, or ROM. While the volatile memory described herein may be referred to as RAM, any volatile memory that would not maintain data stored therein, once unplugged from a host and/or power, would be appropriate. The memory 1542 and the additional storage 1546, both removable and non-removable, are both additional examples of non-transitory computer-readable storage media.

The service provider computer 1502 may also contain communications connection(s) 1548 that allow the service provider computer 1502 to communicate with a data store, another computing device or server, user terminals and/or other devices via the network 1508. The service provider computer 1502 may also include I/O device(s) 1550, such as a keyboard, a mouse, a pen, a voice input device, a touch input device, a display, speakers, a printer, etc.

Turning to the contents of the memory 1542 in more detail, the memory 1542 may include an operating system 1552 and/or one or more application programs or services for implementing the features disclosed herein including a provisioning engine(s) 1541 (e.g., transport services 210 and 244, provisioning service 224, transaction processing service 226 and 254, and/or authentication service 230).

The various examples further can be implemented in a wide variety of operating environments, which in some cases can include one or more user computers, computing devices or processing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system also can include a number of workstations running any of a variety of commercially-available operating systems and other known applications for purposes such as development and database management. These devices also can include other electronic devices, such as dummy terminals, thin-clients, gaming systems, and other devices capable of communicating via a network.

Most examples utilize at least one network that would be familiar to those skilled in the art for supporting communications using any of a variety of commercially available protocols, such as TCP/IP, OSI, FTP, UPnP, NFS, CIFS, and AppleTalk. The network can be, for example, a local area network, a wide-area network, a virtual private network, the Internet, an intranet, an extranet, a public switched telephone network, an infrared network, a wireless network, and any combination thereof.

In examples utilizing a network server, the network server can run any of a variety of server or mid-tier applications, including HTTP servers, FTP servers, CGI servers, data servers, Java servers, and business application servers. The server(s) may also be capable of executing programs or scripts in response to requests from user devices, such as by executing one or more applications that may be implemented as one or more scripts or programs written in any programming language, such as Java®, C, C# or C++, or any scripting language, such as Perl, Python or TCL, as well as combinations thereof. The server(s) may also include database servers, including without limitation those commercially available from Oracle®, Microsoft®, Sybase®, and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of examples, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen, or keypad), and at least one output device (e.g., a display device, printer, or speaker). Such a system may also include one or more storage devices, such as disk drives, optical storage devices, and solid-state storage devices such as RAM or ROM, as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a non-transitory computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or browser. It should be appreciated that alternate examples may have numerous variations from that described above. For example, customized hardware might also be used and/or particular elements might be implemented in hardware, software (including portable software, such as applets) or both. Further, connection to other computing devices such as network input/output devices may be employed.

Non-transitory storage media and computer-readable media for containing code, or portions of code, can include any appropriate media known or used in the art, including storage media, such as, but not limited to, volatile and non-volatile, removable and non-removable media implemented in any method or technology for storage of information such as computer-readable instructions, data structures, program modules, or other data, including RAM, ROM, EEPROM, flash memory or other memory technology, CD-ROM, DVD or other optical storage, magnetic cassettes, magnetic tape, magnetic disk storage or other magnetic storage devices, or any other medium which can be used to store the desired information and which can be accessed by a system device. Based at least in part on the disclosure and teachings provided herein, a person of ordinary skill in the art will appreciate other ways and/or methods to implement the various examples.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated examples thereof are shown in the drawings and have been described above in detail.

It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “having,” “including,” and “containing” are to be construed as open-ended terms (e.g., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate examples of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Preferred examples of this disclosure are described herein, including the best mode known to the inventors for carrying out the disclosure. Variations of those preferred examples may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

As described above, one aspect of the present technology is the gathering and use of data available from various sources to provide a comprehensive and complete window to a user's personal health record. The present disclosure contemplates that in some instances, this gathered data may include personally identifiable information (PII) data that uniquely identifies or can be used to contact or locate a specific person. Such personal information data can include demographic data, location-based data, telephone numbers, email addresses, Twitter ID's, home addresses, data or records relating to a user's health or level of fitness (e.g., vital sign measurements, medication information, exercise information), date of birth, health record data, or any other identifying or personal or health information.

The present disclosure recognizes that the use of such personal information data, in the present technology, can be used to the benefit of users. For example, the personal information data can be used to provide enhancements to a user's personal health record. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the U.S., collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services or other services relating to health record management, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

What is claimed is:

1. A computer-implemented method, comprising:
receiving, from a first user device associated with a first user account, a request to establish a restricted mobile payment account on behalf of a second user account, the restricted mobile payment account to be a subaccount of a primary mobile payment account of the first user account, the request comprising user account information that is associated with the second user account;
determining that the second user account is included in a user account group with the first user account based at least in part on the user account information;
sending a registration notification to a second user device to trigger registration of the restricted mobile payment account with a payment service, the second user device associated with the second user account;
receiving, from the second user device and based at least in part on the registration notification, a registration request that comprises a unique user account identifier of the second user account;
causing creation of the restricted mobile payment account based at least in part on the user account information and the unique user account identifier of the second user account; and
registering the first user account to receive transaction activity notifications corresponding to transactions of the restricted mobile payment account.

2. The computer-implemented method of claim 1, wherein registering the first user account to receive the transaction activity notifications comprises sharing a token and a resource locator with the first user device, the resource locator identifying a network location of a transaction activity table that stores the transactions of the restricted mobile payment account, and the token being usable to access the transaction activity table.

3. The computer-implemented method of claim 1, further comprising:
receiving, from an issuing system, an indication of a new transaction of the restricted mobile payment account;
sending, to the first user device and based at least in part on the indication, a transaction activity notification corresponding to the new transaction;
receiving, from the first user device, a request for transaction information corresponding to the new transaction; and
sending, to the first user device and based at least in part on the request, the transaction information.

4. The computer-implemented method of claim 3, wherein the request for the transaction information comprises a token and a resource locator, and wherein the method further comprises:
accessing a transaction activity table at a resource location identified by the resource locator; and
using the token to read the transaction information in the transaction activity table.

5. The computer-implemented method claim 1, wherein the user account information is obtained from a remote storage and compute system or input at the first user device.

6. The computer-implemented method of claim 1, wherein the user account group comprises an owner role and at least one of a participant role or a managing participant role.

7. The computer-implemented method of claim 6, and wherein the first user account is the owner role and the second user account is the participant role.

8. One or more computer-readable media comprising computer-executable instructions that, when executed by one or more processors, cause the one or more processors to perform operations comprising:
receiving, from a first user device associated with a first user account, a request to establish a restricted mobile payment account on behalf of a second user account, the restricted mobile payment account to be a subaccount of a primary mobile payment account of the first user account, the request comprising user account information that is associated with the second user account;
sending a registration notification to a second user device to trigger registration of the restricted mobile payment account with a payment service, the second user device associated with the second user account;
receiving, from the second user device and based at least in part on the registration notification, a registration request that comprises a unique user account identifier of the second user account;
causing creation of the restricted mobile payment account based at least in part on the user account information and the unique user account identifier of the second user account; and
registering the first user account to receive transaction activity notifications corresponding to transactions of the restricted mobile payment account.

9. The one or more computer-readable media of claim 8, wherein the computer-executable instructions further cause the one or more processors to perform operations comprising determining that the second user account is included in a user account group with the first user account based at least in part on the user account information, the user account group comprising a set of user accounts that share account services and account privileges.

10. The one or more computer-readable media of claim 9, wherein the user account group comprises an owner role and at least one of a participant role or a managing participant role.

11. The one or more computer-readable media of claim 9, wherein the computer-executable instructions further cause the one or more processors to perform operations comprising registering a third user account to receive the transaction activity notifications, the third user account included in the user account group.

12. The one or more computer-readable media of claim 8, wherein the payment service comprises a peer-to-peer payment service.

13. The one or more computer-readable media of claim 8, wherein causing creation of the restricted mobile payment account comprises:
sending an account creation request to an external issuing system; and
receiving a confirmation of execution of the account creation request, the confirmation comprising a payment account identifier of the restricted mobile payment account.

14. The one or more computer-readable media of claim 8, wherein the computer-executable instructions further cause the one or more processors to perform operations comprising storing the user account information included in the request to establish the restricted mobile payment account on behalf of the second user account.

15. The one or more computer-readable media of claim 8, wherein the computer-executable instructions further cause the one or more processors to perform operations comprising communicating with an account verification system to verify the second user account based at least in part on the user account information.

16. The one or more computer-readable media of claim 8, wherein the computer-executable instructions further cause the one or more processors to perform operations comprising converting the restricted mobile payment account to a new primary mobile payment account of the second user account based at least in part on detecting that an age associated with the second user account meets or exceeds an age threshold.

17. The one or more computer-readable media of claim 16, wherein the computer-executable instructions further cause the one or more processors to perform operations comprising, prior to converting the restricted mobile payment account:
presenting a conversion option at a user interface of the second user device based at least in part on detecting that the age associated with the second user account meets or exceeds the age threshold; and
responsive to a selection of the conversion option at the user interface, verifying an identity of a user of the second user account, wherein converting the restricted mobile payment account is based at least in part on verifying the identity of the user of the second user account.

18. A user device, comprising:
a memory comprising computer-executable instructions; and
a processor configured to access the memory and execute the computer-executable instructions to at least:
detect that an age associated with a first user account meets or exceeds an age threshold, the first user account associated with a restricted mobile payment account that is a subaccount of a primary account of a second user account;
present a conversion option at a user interface of the user device based at least in part on detecting that the age associated with the first user account meets or exceeds the age threshold, the conversion option enabling conversion of the restricted mobile payment account to a new primary mobile payment account of the first user account;
responsive to a selection of the conversion option at the user interface, communicate with a payment service to verify an identity of a user of the first user account; and
receive a communication indicating successful creation of the new primary mobile payment account.

19. The user device of claim 18, wherein communicating with the payment service comprises:
providing device metadata to the payment service to verify the age; and
responsive to a request from the payment service, providing user account information to the payment service to verify additional user account details.

20. The user device of claim 18, wherein the processor is further configured to access the memory and execute the computer-executable instructions to at least:
receive a transaction activity notification corresponding to a new transaction of the new primary mobile payment account;
send a request for transaction information corresponding to the new transaction; and
receive, based at least in part on the request, the transaction information.
